(19) United States
(12) Patent Application Publication
(10) Pub. No.: US 2026/0171237 A1
(43) Pub. Date: Jun. 18, 2026

(54) METHOD FOR CONTROLLING PHYSIOTHERAPY INSTRUMENT, COMPUTING DEVICE AND MEDIUM

(71) Applicant: Chongqing Rob Linka Science And Technology Co., Ltd., Chongqing (CN)

(72) Inventors: Jianhua XU, Chongqing (CN); Wentao CHEN, Chongqing (CN)

(73) Assignee: Chongqing Rob Linka Science And Technology Co., Ltd., Chongqing (CN)

(21) Appl. No.: 19/398,203

(22) Filed: Nov. 24, 2025

(30) Foreign Application Priority Data
Dec. 16, 2024 (CN) 202411850989.5

(51) Int. Cl. G16H 40/67; G16H 20/30; H04L 67/125

(52) U.S. Cl. CPC G16H 40/67; G16H 20/30; H04L 67/125

(57) ABSTRACT

A method for controlling a physiotherapy instrument includes: in response to a sequence number assignment command, sending a first data packet via broadcast; receiving a communication address fed back by at least one host on the basis of the first data packet; on the basis of each communication address, sending a respective second data packet to each host of the at least one host; receiving a sequence number assignment result fed back by each host of the at least one host on the basis of a respective target sequence number, the sequence number assignment result including a host sequence number; and on the basis of each sequence number assignment result, associating each communication address with a corresponding host sequence number, and determining a control state corresponding to each communication address.

### CROSS-REFERENCE TO RELATED APPLICATION

This application claims priority to Chinese Patent Application No. 202411850989.5, filed on Dec. 16, 2024, titled “METHOD FOR CONTROLLING PHYSIOTHERAPY INSTRUMENT, COMPUTING DEVICE AND MEDIUM”, which is incorporated herein by reference in its entirety.

### TECHNICAL FIELD

The present disclosure relates to the field of physiotherapy instrument control technologies, specifically to a method for controlling a physiotherapy instrument, a computing device and a media.

### BACKGROUND

The integration of wireless communication technology with physiotherapy instruments enables wireless operation on the physiotherapy instruments. This separates a control end of a physiotherapy instrument into two distinct parts, where one is a remote control terminal for controlling intensities of pulses and transmission of signals, and the other are a plurality of hosts. Each host is used for receiving a signal transmitted by the remote control terminal so as to generate a pulse signal and transmit same to electrode sheets for massage and physical therapy on a human body.

To facilitate control of the hosts, a traditional approach involves burning a unique program into each host, and a sequence number for each host is hard-coded into the program. That is, each host possesses a permanent and distinct sequence number. However, the burning process is complicated, and after the program is burned, it necessitates visually distinguishing the sequence number of each host and storing same separately. Thus, the approach, which requires hard-coded sequence numbers of the hosts, makes it inconvenient to manage the hosts and for a user to use.

It will be noted that, the information disclosed in the background above is intended solely to enhance understanding of the background of the present disclosure, and therefore may include information that does not constitute prior art known to those skilled in the art.

### SUMMARY

To overcome the problem that the traditional approach is inconvenient for management of hosts and user operation due to hard-coding of sequence numbers of hosts, embodiments of the present disclosure provide a method for controlling a physiotherapy instrument, a computing device and a medium.

In a first aspect, in order to solve the above-described technical problem, embodiments of the present disclosure provide a method for controlling a physiotherapy instrument. The method is applied to a remote control terminal, and includes:
in response to a sequence number assignment command, sending a first data packet via broadcast;
receiving a communication address fed back by at least one host on the basis of the first data packet;
sending a respective second data packet to each host of the at least one host on the basis of each communication address, the second data packet including a target sequence number, the target sequence number being a sequence number in an unoccupied state, and each target sequence number being unique;
receiving a sequence number assignment result fed back by each host of the at least one host on the basis of the target sequence number, the sequence number assignment result including a host sequence number, and the host sequence number representing a sequence number of the host of the at least one host; and
on the basis of each sequence number assignment result, associating each communication address with a respective host sequence number, and determining a control state corresponding to each communication address.

In a second aspect, in order to solve the above-described technical problem, embodiments of the present disclosure further provide a method for controlling a physiotherapy instrument. The method is applied to a host, and includes:
receiving a first data packet sent by a remote control terminal;
sending a communication address to the remote control terminal on the basis of the first data packet;
receiving a second data packet fed back by the remote control terminal on the basis of the communication address, the second data packet including a target sequence number, and the target sequence number being a sequence number in an unoccupied state;
taking the target sequence number as a host sequence number, the host sequence number representing a sequence number of the host; and
generating a sequence number assignment result on the basis of the host sequence number, and sending the sequence number assignment result to the remote control terminal, the sequence number assignment result including the host sequence number.

In a third aspect, in order to solve the above-described technical problem, embodiments of the present disclosure further provide a computing device. The computing device includes a memory, a processor and a program which is stored in the memory and executable by the processor. When the processor executes the program, the steps of the method for controlling the physiotherapy instrument described above are performed.

In a fourth aspect, in order to solve the above-described technical problem, embodiments of the present disclosure further provide a computer-readable storage medium. The computer-readable storage medium stores an instruction. When the instruction is executed on a terminal device, the terminal device is caused to perform the steps of the method for controlling the physiotherapy instrument described above.

The beneficial effects of embodiments of the present disclosure are as follows:
the remote control terminal responds to the sequence number assignment command issued by the user to send the first data packet via broadcast, and receives communication addresses fed back by hosts, so as to send second data packets, which carry different target sequence numbers, to the hosts according to the communication address; then, by receiving sequence number assignment results fed back by the hosts on the basis of respective target sequence numbers, the host sequence number corresponding to each host is determined; Then, on the basis of the host sequence number corresponding to each host, the communication addresses are associated with the respective host sequence numbers, and control states corresponding to the communication addresses are determined. In this way, by assigning the target sequence numbers to the hosts as the sequence numbers of the hosts, and associating the corresponding communication addresses with the host sequence numbers fed back by the hosts, the hosts are assigned the respective sequence numbers. Therefore, there is no need to hard-code the sequence number of each host, which facilitates management of the hosts and user operations.

### BRIEF DESCRIPTION OF THE DRAWINGS

FIG. 1 is a flow diagram of a method for controlling a physiotherapy instrument provided by embodiments of the present disclosure;

FIG. 2 is a flowchart of a method for processing a communication address by a remote control terminal provided by embodiments of the present disclosure;

FIG. 3 is a flowchart of a method for assigning a sequence number to a host by a remote control terminal provided by embodiments of the present disclosure;

FIG. 4 is a flow diagram of another method for controlling a physiotherapy instrument provided by embodiments of the present disclosure;

FIG. 5 is a flowchart of a method for determining a host sequence number by a host provided by embodiments of the present disclosure; . and

FIG. 6 is a diagram of interaction between a remote control terminal and a host provided by embodiments of the present disclosure.

### DETAILED DESCRIPTION

The principle and features of the present disclosure are to be described below. The examples provided herein are intended solely to illustrate the disclosure, but are not intended to limit the scope of the present disclosure.

A physiotherapy instrument includes a remote control terminal, a plurality of hosts, and a plurality of electrode sheets. The remote control terminal is in communication connection with the hosts, and the hosts are connected to the electrode sheets. The remote control terminal is a dedicated remote control for controlling the hosts. The remote control terminal may be an application (APP) for a mobile phone or tablet computer, or may be a dedicated physiotherapy device for massage and physical therapy. The remote control terminal is mainly used for enabling human-machine interaction, thereby facilitating selection of parameters, such as a mode and a duration, of physical therapy by a user for adjustment of physical therapy. The hosts are mainly used for receiving control commands transmitted by the remote control terminal and then generating corresponding pulses. The electrode sheets are used for applied to areas of a human body requiring physical therapy. The hosts apply the generated pulses to the areas of the human body requiring physical therapy via the electrode sheets, thereby providing massage and physical therapy for the user.

A method for controlling a physiotherapy instrument, a computing device and a medium in embodiments of the present disclosure are described hereinafter with reference to the accompanying drawings.

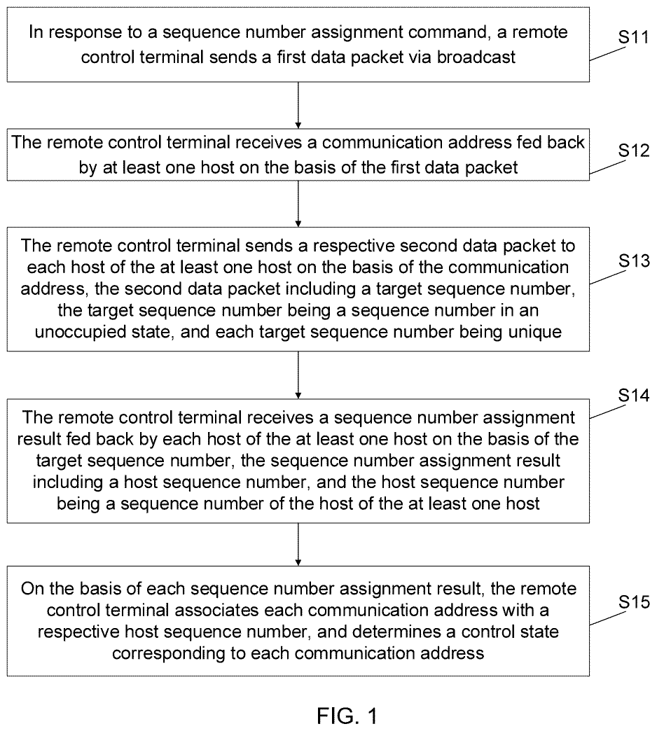

As shown in FIG. 1, embodiments of the present disclosure provide a method for controlling a physiotherapy instrument. The method is applied to a remote control terminal, and includes the following steps.

In step S11, in response to a sequence number assignment command, the remote control terminal sends a first data packet via broadcast. For example, the sequence number assignment command is issued by a user.

In step S12, the remote control terminal receives a communication address fed back by at least one host on the basis of the first data packet.

In some embodiments, the remote control terminal receives the communication address fed back by the at least one host via broadcast. The communication address represents address information of the host.

In step S13, the remote control terminal sends a respective second data packet to each host of the at least one host on the basis of the communication address. Here, the second data packet includes a target sequence number. The target sequence number is a sequence number in an unoccupied state. Each target sequence number is unique.

In some embodiments, an occupancy state includes an occupied state and an unoccupied state. A sequence number being in an unoccupied state indicates that the sequence number has not been assigned to any host. A sequence number being in an occupied state indicates that the sequence number has been assigned to a host.

In step S14, the remote control terminal receives a sequence number assignment result fed back by each host of the at least one host on the basis of the target sequence number. Here, the sequence number assignment result includes a host sequence number. The host sequence number is a sequence number of the host of the at least one host. That is, the host sequence number is a sequence number assigned to the host by the remote control terminal.

In step S15, on the basis of each sequence number assignment result, the remote control terminal associates each communication address with a respective host sequence number, and determines a control state corresponding to each communication address.

Here, associating the communication address with the respective host sequence number represents establishing a link between the communication address and the respective host sequence number. That is, the remote control terminal can locate the respective communication address by means of the host sequence number; conversely, the remote control terminal can find the host sequence number by means of the communication address. Thus, the remote control terminal can control the host corresponding to the communication address. For example, the remote control terminal associates communication address a with host sequence number 1. In this case, once the remote control terminal locates communication address a associated with host sequence number 1, the remote control terminal can send a relevant control command to a host pointed to by host sequence number 1 by means of communication address a, thereby achieving control of the host with host sequence number 1.

For ease of understanding, embodiments of the present disclosure illustrate a process by which a remote control terminal assigns target sequence number 2 to Host A as follows. The remote control terminal sends a second data packet to Host A, a target sequence number in the second data packet being 2. The host takes the target sequence number as a host sequence number, that is, Host A takes 2 as a host sequence number; and on the basis of the host sequence number, Host A generates a sequence number assignment result, and sends same to the remote control terminal. The remote control terminal associates a communication address of Host A with the host sequence number in the sequence number assignment result, that is, the remote control terminal associates the communication address of Host A with host sequence number 2. In this manner, when the remote control terminal is to send a control command to the host with host sequence number 2, the remote control terminal first locates the communication address associated with host sequence number 2, and then sends the control command to the associated communication address. Thus, transmission of the control command to the host with host sequence number 2 is achieved.

In some embodiments, a control state includes a connected control state and an unconnected control state. When a control state corresponding to a communication address is a connected control state, it indicates that the remote control terminal has assigned a sequence number to a host pointed to by the communication address, that is, the remote control terminal has associated the communication address with a respective host sequence number. When a control state corresponding to a communication address is an unconnected control state, it indicates that the remote control terminal has not assigned a sequence number to a host pointed to by the communication address, that is, the remote control terminal has not associated the communication address with a respective host sequence number.

In the method for controlling the physiotherapy instrument provided in the embodiments of the present disclosure, the remote control terminal responds to the sequence number assignment command issued by the user to send the first data packet via broadcast, and receives the communication address fed back by each host, so as to send the second data packet carrying the unique target sequence number to each host according to the communication address. Then, by receiving the sequence number assignment result fed back by each host on the basis of the target sequence number, the host sequence number corresponding to each host is determined. Then, on the basis of the host sequence number corresponding to each host, each communication address is associated with the respective host sequence number, and the control state corresponding to each communication address is determined. In this way, by assigning the target sequence number to the host as the sequence number of the host, and associating the corresponding communication addresses with the host sequence number fed back by the host, each host is assigned the respective sequence number. Therefore, there is no need to hard-code a sequence number for each host, which facilitates management of hosts and user operations.

In this case, during user operations, by means of communication between the remote control terminal and the hosts, the sequence number assignment command can be issued via the remote control terminal, and thus the unique host sequence number is assigned to each host by using the remote control terminal. That is, sequence numbers are randomly assigned to the hosts. When there are a plurality of hosts, a first host, a second host, . . . , and an Nth host can be determined by the method for controlling the physiotherapy instrument provided in the embodiments of the present disclosure, thereby enabling a user to send and manage different control commands between the plurality of hosts.

In some embodiments, the step of sending the respective second data packet to each host of the at least one host on the basis of each communication address includes: determining whether each communication address successfully matches any of communication addresses stored in a preset pairing data table; if so, determining each successfully matching communication address as a first target communication address, and determining a host pointed to by the first target communication address as a first target host; and sending a respective second data packet to each first target host on the basis of each first target communication address.

The remote control terminal sends the first data packet via broadcast. Each physiotherapy instrument includes a remote control terminal and hosts paired with the remote control terminal. However, within a common environment, there may be a plurality of physiotherapy instruments, which leads to a situation where a host not belonging to a same physiotherapy instrument as a remote control terminal may also feed back a communication address to the remote control terminal. In this case, each communication address is matched against the communication addresses stored in the preset pairing data table, and a successful match indicates that a host corresponding to the communication address is a host paired with the remote control terminal, that is, the communication address is a first target address. Then, by sending a respective second data packet to a first target host pointed to by each first target communication address, it can be ensured that the remote control terminal sends second data packets carrying target sequence numbers exclusively to hosts paired with the remote control terminal, so that the remote control terminal assigns sequence numbers solely to the hosts paired therewith, thereby preventing management confusion and facilitating management of the hosts.

In some embodiments, each remote control terminal corresponds to one preset pairing data table. The preset pairing data table stores therein communication addresses of hosts paired with the remote control terminal. The remote control terminal performs a table lookup operation on each received communication address within the corresponding preset pairing data table. If a communication address identical to the received communication address is found, it is determined that the match for the communication address received by the remote terminal is successful.

In some embodiments, the step of sending the respective second data packet to the first target host on the basis of each first target communication addresses includes: determining a control state corresponding to each first target communication address; determining a first target communication address, whose control state is an unconnected control state, as a second target communication address; determining a host pointed to by the second target communication address as a second target host; and sending a respective second data packet to each second target host on the basis of each second target communication address. Here, a control state includes a connected control state and an unconnected control state.

When a control state corresponding to a communication address is a connected control state, it indicates that the remote terminal has assigned a sequence number to a host pointed to by the communication address. When a control state corresponding to a communication address is an unconnected control state, it indicates that the remote terminal has not assigned a sequence number to a host pointed to by the communication address. Therefore, by determining the control state corresponding to each first target communication address, it may be possible to determine whether the remote control terminal has assigned a sequence number to the first target host pointed to by the first target communication address. If the remote control terminal has not assigned a sequence number to the first target host pointed to by the first target communication address, the remote control terminal then sends, on the basis of the second target communication address, the second data packet carrying the respective target sequence number to each second target host, so as to assign a sequence number to the second target host. In this way, the method enables assignment of a sequence number to a host that lacks one, while also preventing a single host from being assigned multiple sequence numbers, i.e., preventing one host occupying multiple sequence numbers, thereby facilitating control of hosts by the remote control terminal.

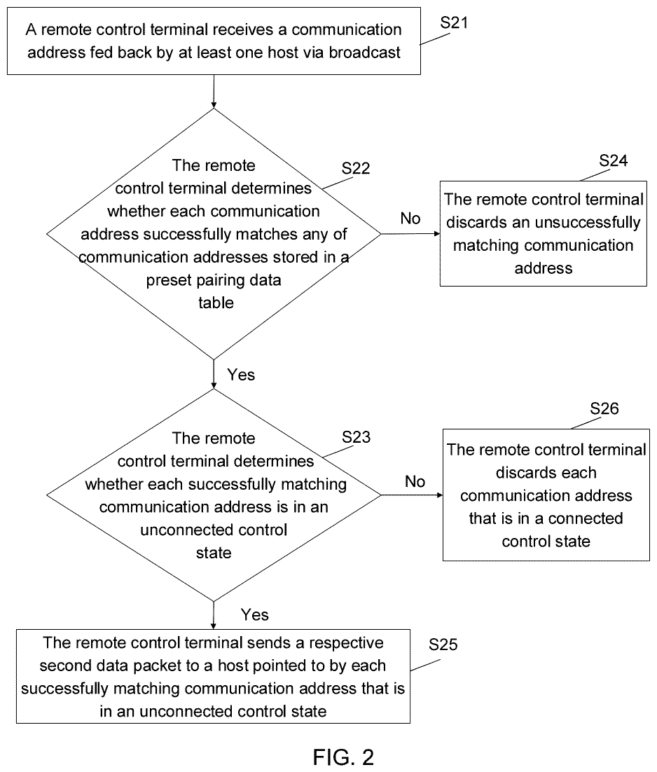

With reference to FIG. 2, embodiments of the present disclosure provide an implementation in which a remote control terminal processes a communication address. The implementation involves the following steps.

In step S21, the remote control terminal receives a communication address fed back by at least one host via broadcast. Here, the communication address is an address, where the host is located, determined by the host on the basis of the first data packet.

In step S22, the remote control terminal determines whether each communication address successfully matches any of communication addresses stored in a preset pairing data table; if so, step S23 is executed; and if not, step S24 is executed. It will be understood that, a successful match indicates that a communication address is present in the preset pairing data table, and thus the communication address is a first target communication address.

In step S23, the remote control terminal determines whether each successfully matching communication address is in an unconnected control state; if so, step S25 is executed; and if not, step S26 is executed.

In step S24, the remote control terminal discards an unsuccessfully matching communication address. It will be understood that, the remote control terminal discards the unsuccessfully matching communication address indicates that the remote control terminal does not respond to the unsuccessfully matching communication address.

In step S25, the remote control terminal sends a respective second data packet to a host pointed to by each successfully matching communication address that is in an unconnected control state. It will be understood that, the communication address in an unconnected control state in this step is a second target communication address, and the host pointed to by the second target communication address is a second target host.

In step S26, the remote control terminal discards each communication address that is in a connected control state.

In some embodiments, before sending the respective second data packet to each host of the at least one host on the basis of each communication address, the method further includes: querying an occupancy state of each sequence number; and determining each sequence number in an unoccupied state as a target sequence number. Here, the occupancy state includes an occupied state and an unoccupied state.

A sequence number being in an unoccupied state indicates that the sequence number has not been assigned to any host. A sequence number being in an occupied state indicates that the sequence number has been assigned to a host. In this way, by querying the occupancy state of each sequence number, and taking each sequence number in an unoccupied state as the target sequence number, it ensures that each target sequence number has not been assigned to any other host, thereby guaranteeing a one-to-one correspondence between hosts and sequence numbers.

In some embodiments, the at least one host includes a plurality of hosts. The step of receiving the communication address fed back by the at least one host on the basis of the first data packet includes: receiving communication addresses fed back by the plurality of hosts on the basis of the first data packet. The step of taking each sequence number in the unoccupied state as the target sequence number includes: taking each sequence number in the unoccupied state as the target sequence number; and determining target sequence numbers pointed to by the communication addresses according to a chronological order in which the communication addresses are received.

Since the remote control terminal sends the first data packet via broadcast, multiple hosts may simultaneously feed back their respective communication addresses within a short time. In this case, by determining a specific value of the target sequence number pointed to by each communication address according to the chronological order in which the communication addresses are received, it is ensured that a single target sequence number is assigned to only one host, thereby preventing confusion of sequence numbers of hosts and facilitating management.

In some embodiments, the target sequence numbers are assigned in ascending order to the communication addresses in a sequence of their reception. For example, the remote control terminal receives respective second target communication addresses at 10:01, 10:02 and 10:03 on a same day; and target sequence numbers include 1, 2 and 3. Thus, according to a sequence in which the second target communication addresses are received, a target sequence number pointed to by a communication address received at 10:01 is determined to be 1, a target sequence number pointed to by a communication address received at 10:02 is determined to be 2, and a target sequence number pointed to by a communication address received at 10:03 is determined to be 3.

Furthermore, the step of receiving the sequence number assignment result fed back by each host on the basis of the target sequence number includes: receiving a sequence number assignment result fed back by each second target host on the basis of the target sequence number. In this way, by receiving the sequence number assignment result fed back by each second target host, a sequence number corresponding to each second target host can be determined at a remote control terminal side.

Furthermore, the sequence number assignment result further includes a communication address of the host. The step of associating each communication address with the respective host sequence number on the basis of each sequence number assignment result and determining the control state corresponding to each communication address includes: determining whether a communication address corresponding to each sequence number assignment result successfully matches any of the communication addresses stored in the preset pairing data table; and if so, associating each communication address with the respective host sequence number and determining the control state of each communication address.

In this way, by verifying whether the communication address corresponding to each sequence number assignment result is present in the preset pairing data table, interference from non-paired hosts to the remote control terminal may be reduced, thereby facilitating assignment of sequence numbers by the remote control terminal to the hosts paired with the remote control terminal.

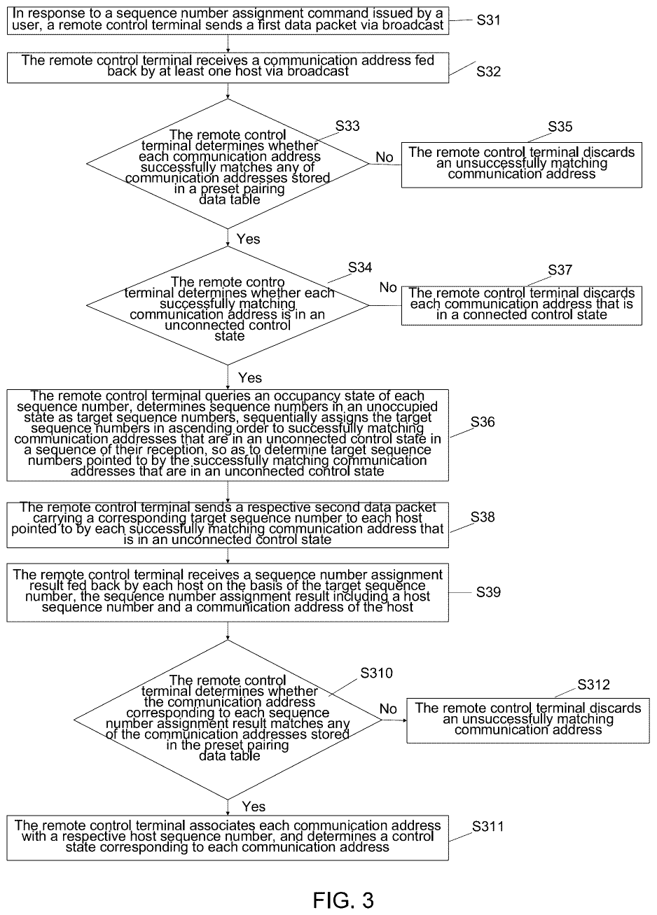

With reference to FIG. 3, embodiments of the present disclosure provide an implementation in which a remote control terminal assigns a sequence number to a host. The implementation involves the following steps.

In step S31, in response to a sequence number assignment command issued by a user, the remote control terminal sends a first data packet via broadcast.

In step S32, the remote control terminal receives a communication address fed back by at least one host via broadcast. Here, the communication address is an address, where the host is located, determined by the host on the basis of the first data packet.

In step S33, the remote control terminal determines whether each communication address successfully matches any of communication addresses stored in a preset pairing data table; if so, step S34 is executed; and if not, step S35 is executed. It will be understood that, a successful match indicates that the communication address is present in the preset pairing data table, and thus the communication address is a first target communication address.

In step S34, the remote control terminal determines whether each successfully matching communication address is in an unconnected control state; if so, step S36 is executed; and if not, step S37 is executed.

In step S35, the remote control terminal discards an unsuccessfully matching communication address.

In step S36, the remote control terminal queries an occupancy state of each sequence number, determines sequence numbers in an unoccupied state as target sequence numbers, sequentially assigns the target sequence numbers in ascending order to successfully matching communication addresses that are in an unconnected control state in a sequence of their reception, so as to determine target sequence numbers pointed to by the successfully matching communication addresses that are in an unconnected control state. Then, step S38 is executed.

In step S37, the remote control terminal discards each communication address that is in a connected control state.

In step S38, the remote control terminal sends a respective second data packet carrying a corresponding target sequence number to each host pointed to by each successfully matching communication address that is in an unconnected control state. Then, step S39 is executed.

In step S39, the remote control terminal receives a sequence number assignment result fed back by each host on the basis of the target sequence number. Here, the sequence number assignment result includes a host sequence number and a communication address of the host. Then, step S310 is executed.

In step S310, the remote control terminal determines whether the communication address corresponding to each sequence number assignment result matches any of the communication addresses stored in the preset pairing data table; if so, step S311 is executed; and if not, step S312 is executed.

In step S311, the remote control terminal associates each communication address with a respective host sequence number, and determines a control state corresponding to each communication address.

In step S312, the remote control terminal discards an unsuccessfully matching communication address.

In some embodiments, the step of associating each communication address with the respective host sequence number and determining the control state corresponding to each communication address includes: associating each communication address with the respective host sequence number, and setting the control state of each communication address associated with the host sequence number to be in a connected control state.

In this manner, by associating communication addresses with host sequence numbers on the remote control terminal side, sequence numbers are assigned to hosts; and by setting the control state of each communication address associated with the host sequence number to be in a connected control state, duplicate sequence number assignments to a single host may be avoided, thereby facilitating control of hosts by the remote control terminal.

In some embodiments, the method, which is applied to the remote control terminal, for controlling the physiotherapy instrument further includes: receiving operational information sent by a host corresponding to each communication address whose control state is a connected control state; and synchronizing data with each host on the basis of the operational information.

In this way, by synchronizing operational data of hosts with the remote control terminal, it is conducive to monitoring an operational condition of each host by the user by means of the remote control terminal, thereby facilitating physical therapy and massage of the user by means of the physiotherapy instrument.

In some embodiments, the operational information includes data such as a current mode and a current intensity of the host.

In some embodiments, the method, which is applied to the remote control terminal, for controlling the physiotherapy instrument further includes: in response to a control command issued by the user and on the basis of a host sequence number corresponding to the control command, sending the control command to a corresponding host, so as to trigger the host to operate on the basis of the control command.

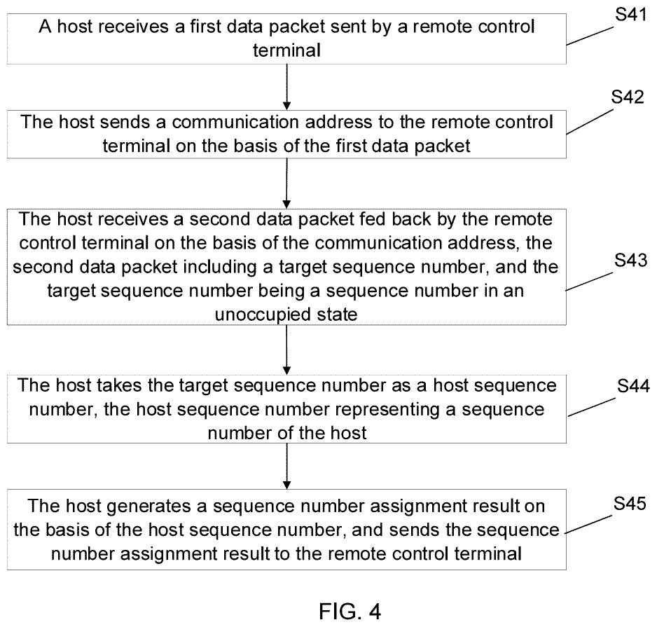

With reference to FIG. 4, embodiments of the present disclosure provide a method for controlling a physiotherapy instrument. The method is applied to a host, and includes the following steps.

In step S41, the host receives a first data packet sent by a remote control terminal.

In step S42, the host sends a communication address to the remote control terminal on the basis of the first data packet.

In step S43, the host receives a second data packet fed back by the remote control terminal on the basis of the communication address. Here, the second data packet includes a target sequence number, and the target sequence number is a sequence number in an unoccupied state.

In step S44, the host takes the target sequence number as a host sequence number. Here, the host sequence number represents a sequence number of the host, i.e., a sequence number assigned by the remote control terminal to the host.

In step S45, the host generates a sequence number assignment result on the basis of the host sequence number, and sends the sequence number assignment result to the remote control terminal, the sequence number assignment result including the host sequence number.

In the method for controlling the physiotherapy instrument provided by the embodiments of the present disclosure, the host feeds back the corresponding communication address to the remote control terminal by means of receiving the first data packet sent by the remote control terminal, so that the remote control terminal feeds back, on the basis of the communication address, the second data packet carrying the target sequence number. Then, the host takes the target sequence number as the host sequence number of a host side, generates the sequence number assignment result, and send the sequence number assignment result carrying the host sequence number to the remote control terminal, so that the remote control terminal associates the communication address of the host with the host sequence number of the host, thereby achieving sequence number assignment. In this way, by receiving corresponding target numbers assigned by the remote control terminal to hosts on the basis of communication addresses of the hosts, and taking by the hosts the target sequence numbers as host sequence numbers, there is no need to hard-coding sequence numbers of the hosts, thereby reducing the need for distinguishing between firmware programs for different hosts, and facilitating management of the hosts and user operations.

In some embodiments, the first data packet includes address information of the remote control terminal. The step of sending the communication address to the remote control terminal on the basis of the first data packet includes: performing pairing on the basis of the address information of the remote control terminal carried in the first data packet; and in a case of successful pairing, sending the communication address to the remote control terminal via broadcast.

In a common operating environment, there may be a plurality of remote control terminals. In this case, after receiving the first data packet, by performing pairing on the address information of the remote control terminal carried in the first data packet, it may be possible to determine whether the first data packet is sent by a remote control terminal paired with the host. If the pairing is successful, it indicates that the first data packet is sent by the remote control terminal paired with the host, and then the communication address of the host is sent to the remote control terminal, so that the remote control terminal paired with the host receives the corresponding communication address.

For example, the host stores therein address information of a remote control terminal belonging to a same physiotherapy instrument as the host, and the remote control terminal is the remote control terminal paired with the host. The host matches the address information of the remote control terminal carried in the first data packet against the address information of the remote control terminal paired with the host device, so as to achieve pairing.

In some embodiments, the second data packet may further include the address information of the remote control terminal. The step of taking the target sequence number as the host sequence number includes: performing pairing on the basis of the address information of the remote control terminal carried in the second data packet; and in a case of a successful pairing, taking the target sequence number as the host sequence number.

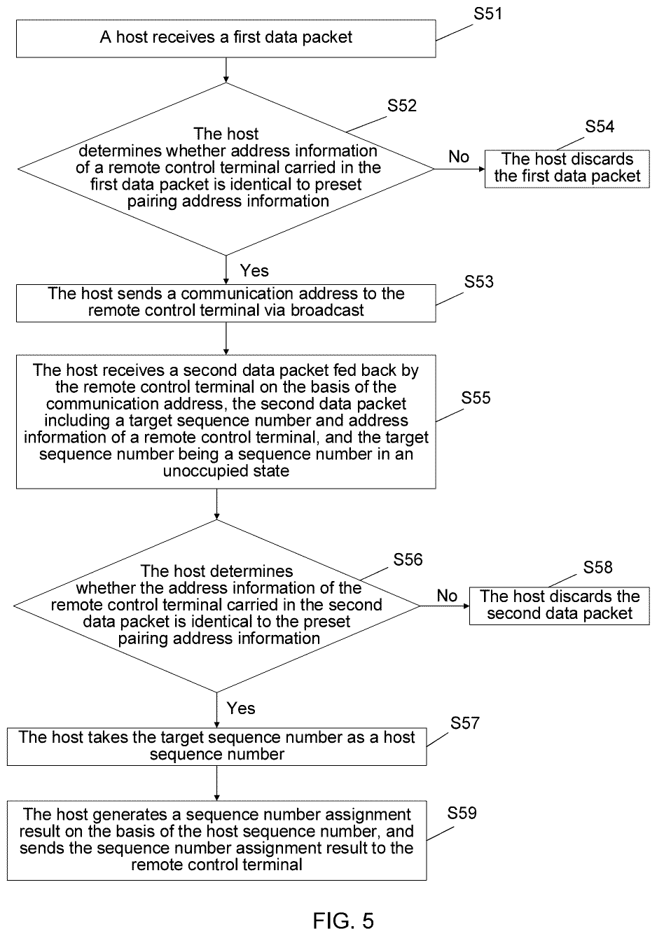

With reference to FIG. 5, embodiments of the present disclosure provide an implementation in which a host determines a host sequence number. The implementation involves the following steps.

In step S51, the host receives a first data packet.

In step S52, the host determines whether address information of a remote control terminal carried in the first data packet is identical to preset pairing address information. If so, step S53 is executed; and if not, step S54 is executed. Here, the preset pairing address information is address information of a remote control terminal belonging to a same physiotherapy instrument as the host.

In step S53, the host sends a communication address to the remote control terminal via broadcast. Then, step S55 is executed.

In step S54, the host discards the first data packet.

In step S55, the host receives a second data packet fed back by the remote control terminal on the basis of the communication address. Here, the second data packet includes a target sequence number and address information of a remote control terminal, and the target sequence number is a sequence number in an unoccupied state. Then, step S56 is executed.

In step S56, the host determines whether the address information of the remote control terminal carried in the second data packet is identical to the preset pairing address information. If so, step S57 is executed; and if not, step S58 is executed.

In step S57, the host takes the target sequence number as the host sequence number. Then, step S59 is executed.

In step S58, the host discards the second data packet.

In step S59, the host generates a sequence number assignment result on the basis of the host sequence number, and sends the sequence number assignment result to the remote control terminal, the sequence number assignment result including the host sequence number.

Furthermore, the method, which is applied to the host, for controlling the physiotherapy instrument further includes: after determining the host sequence number, sending operational information to the remote control terminal that assigns the host sequence number.

In this way, by sending operational information to the remote control terminal that assigns the host sequence number, it is conducive to determining an operational condition of the host by a user by means of the remote control terminal. In some embodiments, the operational information includes parameters such as a mode and an intensity.

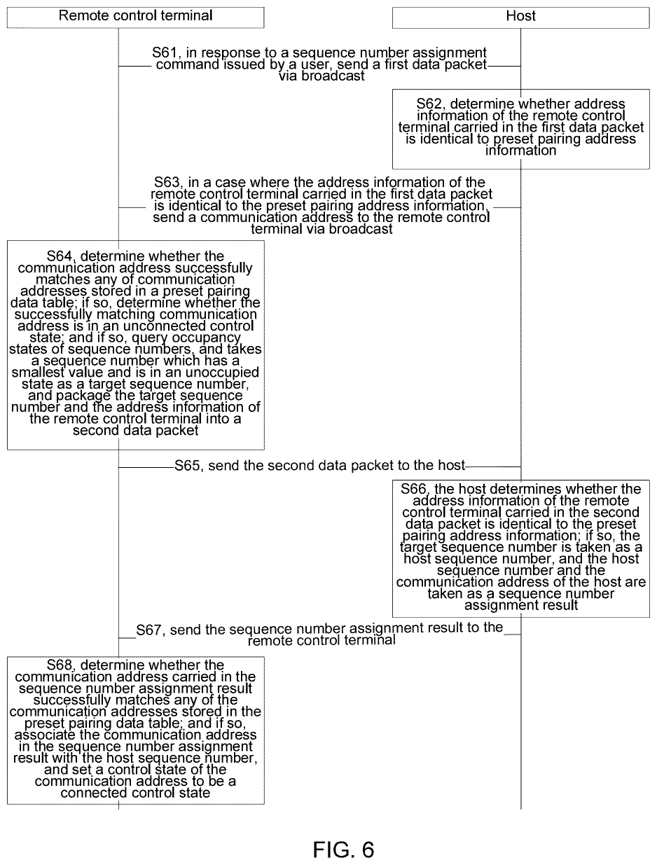

With reference to FIG. 6, embodiments of the present disclosure provide an implementation of interaction between a remote control terminal and a host. For ease of understanding, the implementation involves only one host, and involves the following steps.

In step S61, in response to a sequence number assignment command issued by a user, the remote control terminal sends a first data packet via broadcast. Here, the first data packet carries address information of the remote control terminal.

In step S62, the host determines whether address information of the remote control terminal carried in the first data packet is identical to preset pairing address information.

In step S63, in a case where the address information of the remote control terminal carried in the first data packet is identical to the preset pairing address information, the host sends a communication address to the remote control terminal via broadcast.

In step S64, the remote control terminal determines whether the communication address successfully matches any of communication addresses stored in a preset pairing data table; if so, the remote control terminal determines whether the successfully matching communication address is in an unconnected control state; and if so, the remote control terminal queries occupancy states of sequence numbers, and takes a sequence number which has a smallest value and is in an unoccupied state as a target sequence number, and packages the target sequence number and the address information of the remote control terminal into a second data packet.

In step S65, the remote control terminal sends the second data packet to the host.

In step S66, the host determines whether the address information of the remote control terminal carried in the second data packet is identical to the preset pairing address information; and if so, the target sequence number is taken as a host sequence number, and the host sequence number and the communication address of the host are taken as a sequence number assignment result. The host sequence number represents a sequence number of the host.

In step S67, the host sends the sequence number assignment result to the remote control terminal.

In step S68, the remote control terminal determines whether the communication address carried in the sequence number assignment result successfully matches any of the communication addresses stored in the preset pairing data table; if so, the communication address in the sequence number assignment result is associated with the host sequence number, and a control state of the communication address is set to be a connected control state.

Embodiments of the present disclosure provide a computing device. The computing device includes a memory, a processor and a program which is stored in the memory and executable by the processor. When the processor executes the program, some or all of the steps of the above method for controlling the physiotherapy instrument are performed.

Here, the computing device may be a computer; accordingly, the program is computer software; and for parameters and steps for the computing device in embodiments of the present disclosure, reference may be made to the parameters and the steps in the embodiments for the method for controlling the physiotherapy instrument described above, which will not be elaborated here.

Embodiments of the present disclosure provide a computer-readable storage medium. The computer-readable storage medium stores an instruction. When the instruction is executed, the steps of the method for controlling the physiotherapy instrument described above are performed.

Here, the computer-readable storage medium may be a transitory computer-readable storage medium, or may be a non-transitory computer-readable storage medium.

The technical solutions of the embodiments of the present disclosure may be embodied as a software product. The computer software product is stored on a storage medium, and includes one or more instructions for causing a computer device (which may be a personal computer, a server, a network device, etc.) to execute all or some of the steps of the method in the embodiments of the present disclosure. The computer-readable storage medium may be a non-transitory computer-readable storage medium, including a variety of media capable of storing program codes, such as USB flash drives, portable hard drives, read-only memory (ROM), random access memory (RAM), magnetic disks and optical discs. The computer-readable storage medium may also be a transitory computer-readable storage medium.

Those skilled in the art will recognize that embodiments of the present disclosure may be embodied as a system, a method or a computer program product. Therefore, embodiments of the present disclosure may be embodied in the following forms: complete hardware, complete software (including firmware, resident software, microcode, etc.), or a combination of hardware and software, which is generally referred to as “circuit”, “module” or “system”. Additionally, in some embodiments, the present disclosure may also be embodied as a computer program product in one or more computer-readable media, and the computer-readable media includes computer-readable program codes. The computer-readable storage media may, for example, include but are not limited to electrical, magnetic, optical, electromagnetic, infrared or semiconductor systems, apparatus or devices, or any combination thereof.

In the descriptions of this specification, the reference terms “an embodiment”, “some embodiments”, “an example”, “a specific example”, “some examples”, etc. are intended to indicate that a specific feature, structure, material or characteristic described in conjunction with the embodiment(s) or example(s) is included in at least one embodiment or example of the present disclosure. In this specification, the illustrative expressions of the above-mentioned terms are not necessarily directed to the same embodiment(s)or example(s). In addition, the described specific feature, structure, material or characteristic may be appropriately combined into any one or more embodiments or examples. Furthermore, where not mutually contradictory, those skilled in the art may combine and integrate the various embodiments or examples and the features in different embodiments or examples described in the present specification.

Although embodiments of the present disclosure have been illustrated and described above, it will be understood that, the above embodiments are exemplary but should not be construed as limitations to the scope of the present disclosure. Those skilled in the art may make changes, modifications, substitutions and variations to the above embodiments within the scope of the present disclosure.

What is claimed is:

1. A method for controlling a physiotherapy instrument, the method being applied to a remote control terminal, and comprising:
in response to a sequence number assignment command, sending a first data packet via broadcast;
receiving a communication address fed back by at least one host on the basis of the first data packet;
sending a respective second data packet to each host of the at least one host on the basis of each communication address, wherein the second data packet includes a target sequence number, the sequence number is a sequence number in an unoccupied state, and each target sequence number is unique;
receiving a sequence number assignment result fed back by each host of the at least one host on the basis of the target sequence number, wherein the sequence number assignment result includes a host sequence number, and the host sequence number represents a sequence number of the host of the at least one host; and
associating each communication address with a respective host sequence number on the basis of each sequence number assignment result, and determining a control state corresponding to each communication address.

2. The method according to claim 1, wherein sending the respective second data packet to each host of the at least one host on the basis of each communication address includes:
determining whether each communication address successfully matches any of communication addresses stored in a preset pairing data table;
if so, determining each successfully matching communication address as a first target communication address, and determining a host pointed to by the first target communication address as a first target host; and
sending a respective second data packet to each first target host on the basis of each first target communication address.

3. The method according to claim 2, wherein sending the respective second data packet to each first target host on the basis of each first target communication addresses includes:
determining a control state corresponding to each first target communication address;
determining a first target communication address, whose control state is an unconnected control state, as a second target communication address;
determining a host pointed to by the second target communication address as a second target host; and
sending a respective second data packet to each second target host on the basis of each second target communication address.

4. The method according to claim 1, wherein before sending the respective second data packet to each host of the at least one host on the basis of each communication address, the method further comprises:
querying an occupancy state of each sequence number, wherein the occupancy state includes an occupied state and an unoccupied state; and
determining sequence numbers that are in the unoccupied state as target sequence numbers.

5. The method according to claim 4, wherein the at least one host includes a plurality of hosts;
receiving the communication address fed back by the at least one host on the basis of the first data packet includes:
receiving communication addresses fed back by the plurality of hosts on the basis of the first data packet; and
determining the sequence numbers that are in the unoccupied state as the target sequence numbers, includes:
determining the sequence numbers that are in the unoccupied state as the target sequence numbers; and
determine target sequence numbers pointed to by the communication addresses according to a chronological order in which the communication addresses are received.

6. The method according to claim 1, further comprising:
receiving operational information sent by a host corresponding to each communication address whose control state is a connected control state; and
synchronizing data with each host on the basis of the operational information.

7. The method according to claim 6, wherein the operational information includes:
a current mode of the host corresponding to each communication address whose control state is the connected control state, and
a current intensity of the host corresponding to each communication address whose control state is the connected control state.

8. The method according to claim 1, wherein the sequence number assignment result further includes a communication address of the host of the at least one host; and
associating each communication address with the respective host sequence number on the basis of each sequence number assignment result and determining the control state corresponding to each communication address include:
determining whether a communication address corresponding to each sequence number assignment result successfully matches any of communication addresses stored in a preset pairing data table; and
if so, associating each communication address with the respective host sequence number and determining the control state of each communication address.

9. The method according to claim 1, wherein the control state includes a connected control state and an unconnected control state; and
associating each communication address with the respective host sequence number and determining the control state corresponding to each communication address include:
associating each communication address with the respective host sequence number, and
setting the control state of each communication address associated with the host sequence number to be in the connected control state.

10. The method according to claim 1, further comprising:
in response to a control command and on the basis of a host sequence number corresponding to the control command, sending the control command to a host pointed to by the host sequence number corresponding to the control command, so as to trigger the host pointed to by the host sequence number corresponding to the control command to operate on the basis of the control command.

11. A method for controlling a physiotherapy instrument, the method being applied to a host, and comprising:
receiving a first data packet sent by a remote control terminal;
sending a communication address to the remote control terminal on the basis of the first data packet;
receiving a second data packet fed back by the remote control terminal on the basis of the communication address, wherein the second data packet includes a target sequence number, and the target sequence number is a sequence number in an unoccupied state;
taking the target sequence number as a host sequence number, wherein the host sequence number represents a sequence number of the host;
generating a sequence number assignment result on the basis of the host sequence number, wherein the sequence number assignment result includes the host sequence number; and
sending the sequence number assignment result to the remote control terminal.

12. The method according to claim 11, wherein the first data packet includes address information of the remote control terminal; and
sending the communication address to the remote control terminal on the basis of the first data packet includes:
performing pairing on the basis of the address information of the remote control terminal carried in the first data packet; and
in a case of a successful pairing, sending the communication address to the remote control terminal.

13. The method according to claim 11, wherein the second data packet further includes address information of the remote control terminal; and
taking the target sequence number as the host sequence number includes:
performing pairing on the basis of the address information of the remote control terminal carried in the second data packet; and
in a case of a successful pairing, taking the target sequence number as the host sequence number.

14. The method according to claim 11, further comprising:
after determining the host sequence number, sending operational information to the remote control terminal.

15. The method according to claim 14, wherein the operational information includes a mode of the host and an intensity of the host.

16. A computing device, comprising: a memory, a processor and a program which is stored in the memory and executable by the processor, wherein when the processor executes the program, the method for controlling the physiotherapy instrument according to claim 1 is implemented.

17. A computing device, comprising: a memory, a processor and a program which is stored in the memory and executable by the processor, wherein when the processor executes the program, the method for controlling the physiotherapy instrument according to claim 11 is implemented.

18. A computer-readable storage medium, storing an instruction, wherein when the instruction is executed on a terminal device, the terminal device is caused to implement the method for controlling the physiotherapy instrument according to claim 1.

19. A computer-readable storage medium, storing an instruction, wherein when the instruction is executed on a terminal device, the terminal device is caused to implement the method for controlling the physiotherapy instrument according to claim 11.
